(19) United States
(12) Patent Application Publication
(10) Pub. No.: US 2001/0039007 A1
(43) Pub. Date: Nov. 8, 2001

(54) METHODS AND COMPOSITIONS FOR DETERMINING LATENT VIRAL LOAD

(76) Inventors: ROBERT HALLOWITZ, GAITHERSBURG, MD (US); VIRGINIA SALAS, ALBUQUERQUE, NM (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 09/296,534

(22) Filed: Apr. 22, 1999

Related U.S. Application Data
(63) Continuation-in-part of application No. 09/139,633, filed on Aug. 25, 1998, now Pat. No. 6,127,490.
(63) Continuation of application No. PCT/US97/18649, filed on Oct. 15, 1997.
(63) Continuation-in-part of application No. 08/732,782, filed on Oct. 15, 1996, now Pat. No. 5,817,458.
Application No. 08/732,782 is a continuation-in-part of application No. 08/732,784, filed on Oct. 15, 1996, now Pat. No. 5,714,390.

(51) Int. Cl. C12Q 1/00; C12Q 1/70

(52) U.S. Cl. 435/5; 435/4

(57) ABSTRACT

The present invention relates to a new HIV status of a patient called “latent viral load.” To measure the “latent viral load,” in accordance with a preferred embodiment of the present invention, a population of sample cells is obtained from a desired source, such as an infected patient. The sample cell population is depleted of overtly infected cells and cells harboring active virus, to produce a subset of “resting cells” comprising uninfected and latently-infected cells. This subset is treated with an agent and/or condition that activates the latent virus in the host cell genome and results in a productive infection. The thus-produced infection reflects the “latent viral load” of the host because it reveals the presence of quiescent virus in cells. The latent viral load is useful in assessing a patient's disease status and the efficacy of highly active antiretroviral therapy and other treatment protocols.

This application is a continuation-in-part of 09/139,633 filed Aug. 25, 1998 which is a 371 of PCT/US97/18649, filed Oct. 15, 1997, which is a continuation-in-part of U.S. Ser. No. 08/732,782, filed Oct. 15, 1996, now U.S. Pat. No. 5,817,458, and U.S. Ser. No. 08/732,784, filed Oct. 15, 1996, now U.S. Pat. No. 5,714,390, all of which are incorporated by reference herein.

### BACKGROUND

The ultimate goal of pharmacological treatment for HIV disease is to eradicate the virus. Although promising results began to accrue with the advent of triple therapy composed of nucleoside analogs and protease inhibitors, further study revealed that the virus sequestered in lymphoid tissues had achieved a stable latency. Stable latency is defined as integrated proviral DNA in the host genome that is not actively generating the constituents required to generate mature virus. In fact, there well may be no external manifestation of surface antigen receptor expression in the latently infected lymphoid cell to distinguish it from its uninfected neighbors.

Studies utilizing in situ hybridization targeting proviral DNA have in fact demonstrated the existence of this state of stable latency. The problem is that once the viral load as measured in the peripheral circulation has been reduced to undetectable levels, even by the most sensitive of methods, the discovery of stable latency means that in order to assess the proximity to total elimination of HIV one has to biopsy lymphoid tissues to quantitate the amount of stably latent infected cells remaining.

The process of PCR driven in situ hybridization is very labor intensive and fraught with difficulties in both specimen handling and execution of the process of labeling proviral DNA. Recently, it has been possible to active latent HIV-1 infected Jurkat cells in vitro using phorbol esters. The HIV-1 stable latency quantitation system as described below provides a means to establish a measure of a new HIV status within a patient. We call this measure the “latent viral load.”

### SUMMARY OF THE INVENTION

The present invention relates to methods, compositions, articles of manufacture, and improvements thereof, for detecting, measuring, and/or quantifying stable viral latency as a new measure of viral status in a host infected with a virus which has integrated into the host genome. Stable viral latency can be generally defined as a state where proviral DNA is integrated into the host genome and is not actively expressing the constituents required to generate mature virus. Cells which have stable latent infections may therefore manifest no overt signs of disease, including no production of cell surface antigens, reverse transcriptase, or other signposts of infection. The stealth-like nature of latently infected cells not only means that an infection can escape detection, but it also means that the efficacy of treatments for the disease can not be truly evaluated. The present invention provides means to establish a measure of a new viral status of a virally-infected host by identifying the presence and/or amount of cells in such host which are latently-infected. This new status is referred as “latent viral load” since it is a measure of the presence of dormant virus in an infected host. The latent viral load is useful for diagnostic and prognostic purposes. For instance, it is especially useful in determining the course of viral treatment by ascertaining the presence and number of cells that are latently infected. Latent viral load can be determined for any virus which can integrate into the host genome, including, e.g., human immunodeficiency virus (“HIV”), SIV, HHV1, HHV2, Varicella zoster, CMV, EBV, HHV6A, HHV6B, HHV7, HHV8, HTLV1, HTLV2, etc. In preferred embodiments of the invention, the virus is HIV, especially HIV-1. Although the examples and discussions may relate to HIV, this is for illustrative purposes only and any of the methods, compositions, etc. can be used with other viruses, such as the aforementioned viruses.

To measure the “latent viral load,” in accordance with a preferred embodiment of the present invention, a population of sample cells is obtained from a desired source, such as an infected patient. The sample cell population is depleted of overtly infected cells and cells harboring active virus, to produce a subset of “resting cells” comprising uninfected and latently-infected cells. This subset is treated with an agent and/or condition that activates the latent virus in the host genome and results in a productive infection. The thus-produced infection reflects the “latent viral load” of the host because it reveals the presence of quiescent virus in cells.

The conversion of latently infected cells to productively infected cells can be measured by any characteristic of active viral infection, e.g., production of infectious virus particles, reverse transcriptase, secreted antigens, cell-surface antigens, soluble antigens, RNA, DNA, etc. Preferably, productive cells are ascertained by measuring an antigen expressed on the surface of the cells. When a cell-surface antigen is used, activation of latent cells can be measured by simply separating out those cells which express the antigen in response to the activation agent. The corresponding latent viral load can be quantified in any useful way, including, by counting the cells which express the viral cell-surface (e.g., as cell number per unit volume), measuring the amount of agent which produces the infection, or any other useful way of expressing it. When the number of cells latently infected with virus is used as the measure of latent viral load, each cell can be referred to as a unit of infectivity since it has the capability of infecting other cells upon activation of the dormant virus.

### DETAILED DESCRIPTION OF THE INVENTION

One aspect of the invention relates to a method of determining the latent viral load in a host infected with a virus comprising: treating latently-infected cells, or cells suspected of being latently infected, obtained from the host with an effective amount of an agent capable of activating the virus when integrated into the genome of the cells; and detecting the expression of cell-surface antigen after the cells have been treated with the agent, wherein the presence and/or amount of cells expressing cell-surface antigen is a measure of latent viral load.

In a highly preferred embodiment of the invention, the following procedure can be used to measure the latent virus load in patients infected with HIV:

1. Obtain a tonsilar or other lymphoid tissue biopsy.

2. Macerate and filter the tissue to produce a suspension of cells, including mononuclear lymphoid cells such as FDC's (follicular dendritic cells), macrophages, monocytes, T-cells (e.g., CD4 and CD8), and B-cells (e.g., CD19).

3. Remove HIV-1 infected cells from the cell suspension based on the expression of a viral antigen on the surface of infected cells, preferably gp120, to produce a depleted population.

4. Separate recently HIV-infected and/or activated cells from the depleted population to produce a resting cell population.

5. Treat the resting cells with phorbol ester, or another suitable viral activating reagent.

6. Isolate and count cells which have been activated or induced to express a viral antigen on the surface of infected cells, preferably gp120, by viral activating agent.

This assay when optimized for phorbol ester, or other activating reagent exposure, will yield a certain quantity of actively HIV producing cells (“productive cells”). This number can be referred to as the latent viral load. A goal of treatment can be to reach zero productive cells in lymphoid tissue, i.e., a latent load of 0.

In accordance with the present invention, the latent viral load can be measured in any cell population. Generally, any cell type which is capable of being infected can be used. Examples of tissues which can be examined for the presence of latently-infected cells include, e.g., lymphoid tissues, such as generative organs, peripheral organs, bone marrow, thymus, lymph nodes, adenoids, spleen, Peyer's patches in the lamina propria of small intestine, tonsils in the pharynx, submucousal lymphoid follicles in the appendix and upper airways; peripheral blood; brain and the central nervous system; and any other tissues suspected of harboring latently infected cells.

Tissue samples can be obtained or isolated in any suitable manner. Peripheral blood can be used whole, or fractionated, e.g., by apheresis to enrich for mononuclear cells. Excisional biopsies of a desired tissue, such as a superficial lymph node or tonsil, can be performed conventionally using standard surgical procedures. For many purposes, it may be desirable to prepare single cell suspensions of such tissues. These suspensions can be produced according to various methods, including by maceration and filtration of the tissue to produce cell suspensions; digestion with extracellular matrix and other like enzymes; etc.

Typically, the cells which are evaluated for the presence of latent virus are latently-infected cells, or a cell population suspected of being latently infected. In cells which are latently infected, the genome contain integrated proviral DNA. Post-integration latency can be established when productively infected and activated cells return to a resting G₀ state in which there is minimal amount of transcription of viral genes. See, e.g., Chun et al., Nature Medicine, 1:1284-1290, 1995. The presence of integrated HIV-1 can be detected in cells by carrying out polymerase chain reaction using suitable primers, comprising, e.g., LTR polynucleotide sequences. See, e.g., Chun et al., Proc. Natl. Acad. Sci., 94:13193-13197, 1997. The presence of integrated viral nucleic acid by PCR and other nucleic acid detection methods is not an accurate measure of latent viral load since integrated virus may not be competent to produce an active viral infection, e.g., where it is defective or otherwise incapable of generating active virus.

Preferably, the sample cell population is depleted of activated and/or productively infected cells to result in a population resting cells, including resting lymphoid mononuclear cells. Resting mononuclear cells can be defined as immune system cells that are uninfected with the virus, immune system cells that are latently infected with virus and which contain integrated proviral DNA, and/or mixtures thereof. Such cells can include mononuclear lymphoid cells, including, e.g., lymphocytes, such as T-cells (CD4, CD8, cytolytic, helper), B-cells, natural killer cells; mononuclear phagocytes, such as monocytes, macrophages, epithelioid cells, giant cells, microglia, Kupffer cells, alveolar macrophages; dendritic cells, such as interdigitating dendritic cells, Langerhans cells, or follicular dendritic cells; granulocytes; etc. Resting cells can be obtained as described in, e.g., Chun et al., Nature Medicine, 1:1284-1290, 1995; Chun et al., Nature, 387:183-188, 1997. In general, a latently-infected cell can be any cell which contains the virus in a dormant state, e.g., as integrated into the genome, and which manifest few or no overt or active signs of infection.

To measure the latent viral load in a sample cell population, productively infected cells are preferably removed from it. Elimination of productive cells can be accomplished according to any suitable method. Preferably, as discussed in more detail below, productive cells are recognized and removed from the population using gp120, gp41, or other cell-surface determinants which are characteristic of viral infection. By the term “productive cell,” it is meant a cell which is manufacturing virus, viral antigens, viral nucleic, or other markers of an infectious state.

In preferred embodiments of the present invention, prior to treatment with a viral activating agent or viral activating conditions, cells obtained from a desired source are fractionated into subsets on the basis of one or more differentially-expressed characteristics. In especially preferred embodiments of the invention, the cells can be sorted into two main groups: (1) productively infected cells, including cells which are actively expressing a characteristic of viral infection and cells which are productively infected with virus but which are not yet expressing a viral antigen associated with the infection; and (2) cells which are either latently-infected or not infected at all. The latter group can also be referred to as “resting” cells. Resting cells can serve as a latent cellular reservoir of virus in an infected host and therefore it is of interest to measure the latent viral load in such cells.

The depletion of productively infected cells from a population can be accomplished routinely. A general principle of cell sorting or depletion techniques is to identify a characteristic which is present on target cells, but absent in non-target cells, and use the characteristic to select and deplete the target cells from the population. These methods can be accomplished routinely as described below, using, e.g., affinity chromatography, magnetic bead separation, flow cytometry, fluorescence activated cell sorting, and the like. Either positive or negative selection can be used.

In a preferred example of the present invention, the host is infected with human immunodeficiency virus (HIV) and the tissue sample is a lymphoid tissue which comprises cells involved in the immune response, such as lymphocytes, mononuclear phagocytes, macrophages, and other accessory cells. HIV primarily infects CD4+cells, including helper T-cells and macrophages. However, other cell types can become infected, as well, including CD8+cells. In order to assay the latent viral load, cells that are actively infected with virus can be removed from the lymphoid tissue and the remaining resting cells (see, above) can be subsequently challenged with a viral activating agent in accordance with the present invention to obtain a measure of the latent viral load.

A lymphoid tissue can be isolated from a host by biopsy, e.g., tonsillar tissue or a superficial lymph node. The tissue can be macerated, or otherwise manipulated, to produce a suspension of cells, preferably a single-cell suspension, containing T-cells, B-cells, follicular dendritic cells, etc. The next objective is to remove cells from the suspension which are actively-infected with HIV virus. Any determinant or characteristic of active viral infection can be used to separate cells. However, as explained in more detail below, a highly preferred determinant is an envelope protein which is presented on the surface of infected cells, such as gp120 or gp41. The cells expressing such surface antigens can be treated to expose the antigen in the event the latter is not accessible for antibody or binding reagent binding. For instance, antibodies and other reagents can be used to induce a conformational change that results in exposure of the antigen.

Very generally, the cell suspension can divided into subsets depending upon the desired purpose. As mentioned above, one purpose is eliminate cells from the suspension that are actively infected with virus in order to get an accurate measure of latent viral load. However, other purposes can also be served. For instance, it may be desirable to sort cells into subpopulations to evaluate their respective levels of viral expression and latent viral load. Cells can be sorted on the basis of cell cycle, cell-surface expression of membrane proteins, and the like. Various antigens are known which are associated, or specific-for, different cell types and can form a basis for their separation. These antigens include, cell-surface receptors, TCR, CD molecules, etc. See, e.g., Abbas et al. (1997), Cellular and Molecular Immunology, W. B. Saunders Co., especially, Pages 19, 43, 57, 71, 142, 158, and 463-469. Cells can be separated into any desired subpopulation, including, B-cells, mature B cells, activated B cells, naïve B-cells, memory B-cells, plasma cells, T-cells (CD4 or CD8), activated T-cells, naïve T-cells, memory T-cells, monocytes, neutrophils, natural killer cells, endothelial cells, follicular dendritic cells, marrow cells, myeloid cells, etc.

For measuring HIV in infected patients, it may be desirable to divide T-cells into subsets based upon the expression of differentially expressed proteins, especially cell-surface or membrane proteins. As mentioned, to measure latent viral load, it may be desirable to deplete a tissue of cells that are infected with active virus. Typically, within a short period of infection, T-cells begin to transcribe a variety of genes whose products are associated with functional activation. These include, e.g., cellular proto-oncogenes/transcriptional factor genes; cytokine genes; cytokine receptor genes; cell-surface antigens. The corresponding polypeptide products include, e.g. nuclear binding proteins; cellular oncogenes; cytokines such as IFN-gamma, IL-2, TGF-beta, IL-3, IL-4, IL-5, IL-6, GM-CSF; cytokinin receptors, such as IL-2 receptor; c-myb; transferrin; HLA-DR; and VLA-1. See, e.g., Crabtree, G. R., Science, 243:355-361, 1989. Any such product can be used as a marker to select cells from the population.

Thus, in one embodiment of the present invention, gp120 and/or gp41 can be used to deplete a cell population of substantially all cells productively infected with virus. By the phrase “substantially all,” it is meant, e.g., greater than 90%, preferably 95% or greater, more preferably, 99% or greater. After removal of gp120 and/or gp41 bearing cells, the remaining population contains, e.g., activated cells (e.g., recently-infected cells), infected but quiescent cells harboring integrated proviral DNA, and naïve cells. Activated cells can be removed from the population in several ways, e.g., using any of the above-mentioned markers and/or functional characteristics of the cells.

In one embodiment of the present invention, activated cells can be separated from a population on the basis of expression of HLA-DR, and other antigens associated with T-cell activation, e.g., CD69, CD25, and CD38. See, e.g., Chun et al., Nature Medicine, 1:1284-1290, 1995; Chun et al., Nature, 387:183-188, 1997. Magnetic beads, flow cytometry, or a combination of the two can be used to eliminate activated T-cells. For instance, a sample cell population can be contacted with antibodies to HLA-DR under conditions effective for binding to the antigen. Unbound antibody is removed by washing, and cells bearing antibodies are removed by depletion with magnetic beads conjugated to antibodies that recognize the anti-HLA-DR antibody. The resulting cells can be further labeled with a fluorochrome conjugated to anti-HLA-DR antibodies and sorted by flow cytometry to ensure that all activated cells are eliminated. As many cycles as necessary can be accomplished to eliminate substantially all, e.g., 90% or greater, preferably 95% or greater, more preferably, 99% or greater, such as 99.3% or greater, 99.9% or greater, activated cells from the population. Cells not expressing HLA-DR can be referred to as resting cells. The purity of resting cells can be tested conventionally, e.g., by measuring the number of cells in the S-phase or detecting mRNA for thymidine kinase. The sample can be depleted of cells expressing gp120 and/or gp41 prior to, or after, positive selection for the presence of HLA-DR. The resting cells can be directly challenged with a viral activating agent. Alternatively, the resting cells can be preincubated for a period of time and then tested for gp120/gp41 expression. Such cells can be removed prior to challenge with a viral activating agent.

In another embodiment of the present invention, the CD45 antigen can be used to sort populations for testing for latent viral load. Naive or inactive T-cells express a 200 kD isoform of a cell-surface molecule called CD45 that contains a segment which is coded for by an exon referred to as “A.” This CD45 isoform can be recognized by antibodies that are specific for the A coded segment. Cells bearing the antigen can be referred to as CD45RA cells or RA+ cells. In contract, most active and memory T-cells express a 180 kD isoform of CD45 in which the A exon has been spliced out. This isoform is called CD45RO. See, e.g., Abbas et al. (1997), Cellular and Molecular Immunology, W. B. Sanders Company, especially page 22, 23, 156, and 157; and Chapter 11. The CD45RO isoform is also missing other differentially spliced isoforms, such as the B and C exons. The CD45 isoform and the epitope restricted to it can thus by used as a basis to distinguish activated T-cells and T-memory cells from inactive T-cells, e.g., cells which have not been infected with a virus. Antibodies which are selective for CD45RO or CD45RA cells can be made conventionally, and are commercially available. Antibodies selective for or specific-for 45RO include, e.g., UCHL as described in Terry et al. (1988), Immunology, 64:331. CD45RO is also found on monocytes and granulocytes.

A sample cell population can be depleted of gp120 and/or gp41 (or other envelope antigens) expressing cells. Next, CD45RO+ cells can be depleted from by positive selection using flow cytometry, column chromatography, magnetic beads, etc. For instance, a column can be prepared having a glass bead matrix coated with an antibody specific-for CD45RO. The cell suspension can be loaded on to the column for passage art a predefined flow rate. The “target” CD45RO cells are retained in the matrix. The desired cells are untouched and pass through the column for collection. The resulting population contains activated and memory T-cells. The latter can be depleted of activated cells using HLA-DR, or other markers of activation. The cells can also be incubated under suitable conditions, e.g., for 24-48 hours, to provide an opportunity for cells to continue the maturation cycle which results in expression of gp120 and/or gp41. These expressing cells can be removed from the population prior to challenge with a viral activating agent

Prior to, or following, the CD45RO+ positive selection step, the cell suspension can be subjected to other fractionation, depletion, or enrichment steps. For instance, it may be desirable to enrich for CD4 and/or CD8 bearing cells.

The separation of desired cells can be carried out according to any convention methods, e.g., using a fluorescent activated cell sorting (FACS), magnetic beads, affinity chromatography, panning for adherent cells, etc. Positive or negative selection techniques can be used. For example, cells expressing a cell-surface antigen can be positively separated from a population by using a combination of differential labeling and flow cytometry. Flow cytometers have the ability to sort, or physically separate, particles of interest from a sample. For instance, particles, such as cells, can be labeled with reagents, such as fluorochromes. The particles can be separated by the flow cytometer based on whether the fluorochrome has attached to its surface. Useful fluorochromes, include, fluorescein, phycoerythrin, coumarin, allophycocyanin. cascade blue, red 613, red 670, Quantum red, Hoechst 33342, Hoechst 33258, DAPI, chromomycin A3, propidium iodide, ethidium bromide, acridine orange, rhodamine, etc. Flow cytometry can be performed routinely, e.g., as described in Flow Cytometry, A Practical Approach (1994), ed. Ormerod, M. G., Oxford University Press; Practical Flow Cytometry, 3^{rd} edition, ed. Shapiro, Alan R. Liss, Inc.; Flow Cytometry and Clinical Diagnosis (1994), eds. Keren et al., ASCP Press, Inc.; U.S. Pat. Nos. 5,602,349; 5,675,517; 5,665,557; 5,641,457; and 5,582,982.

Very generally, to accomplish separation, cells can be incubated with one or more binding partners, under conditions in which the binding partner, can attach or bind to the cell-surface. By the term “binding partner,” it is meant any molecule or structure that is capable of selectively binding to a cognate ligand. The binding partner can be directly labeled or indirectly labeled using a “sandwich-type” approach. Cells obtained from a desired source can be sorted by one or more steps in which cells are labeled and then fractionated, using FACS, magnetic beads, affinity chromatography, or other separation steps. Antibodies can be of any type, e.g., polyclonal, monoclonal, recombinant, chimeric, humanized, and can be prepared according to any desired method. See, also, screening recombinant immunoglobulin libraries (Orlandi et al., Proc. Natl. Acad. Sci., 86:3833-3837, 1989; Huse et al., Science, 256:1275-1281, 1989); in vitro stimulation of lymphocyte populations; Winter and Milstein, Nature, 349: 293-299, 1991. The antibodies can also be single chain or FAb fragments. The antibodies can be IgG, subtypes, IgG2a, IgG1, IgM, etc. Antibodies, and immune responses, can also be generated by administering naked DNA See, e.g., U.S. Pat. Nos. 5,703,055; 5,589,466; 5,580,859.

In a preferred aspect of the present invention, active infection can be measured by the expression of a cell surface antigen. In the most preferred embodiments, the cellsurface antigen is gp120 and/or gp41. Preferably, cells expressing the antigen are removed from the cell population. Generally, cells can be labeled directly using one binding partner to a surface molecule, or one or more binding partners, where a first binding partner is specific for a cell-surface molecule and a second binding partner is specific-for the first binding partner. An antibody specific-for gp120 is an example of a first binding partner; gp120 is an example of its cognate ligand for the binding partner. In this case, the antibody specific-for gp120 has a high affinity for gp120 permitting it to selectively attach to it in comparison to other antigens not having gp120 epitopes.

In preferred embodiments of the invention, cells expressing gp120 are isolated by a double-labeling technique. Two antibodies can be used: an antibody specific-for gp120 which is coupled to a capture moiety and a second antibody or binding which is specific-for the capture moiety and which is coupled to a magnetic particle. By the term capture moiety, it is meant any molecule or structure which is capable of recognition and attachment by a binding partner. A function of a capture moiety is to provide a handle for grabbing the object to which it is attached. A capture moiety can be, for instance, a hapten or detectable label, such as a fluorochrome, e.g., FITC, TRITC, R-phycoerythrin, Quantum Red, or Cy3, gold, ferritin, biotin, avidin, streptavidin, green fluorescent protein GFP (Chalfie et al., 1994, Science, 263:802; Cheng et al., 1996, Nature Biotechnology, 14:606; Levy et al., 1996, Nature Biotechnology, 14:610), alkaline phosphatase, peroxidase, HRP, urease, an arbitrary hapten, etc.

A second binding partner which is an antibody specific-for FITC, or, which is a streptavidin molecule is used to grab on to the gp120. The second binding partner is preferably coupled to magnetic beads enabling cells coated with the antibody and binding partner complex to be isolated by applying a magnetic filed thereto.

A magnetic particle (bead, microsphere, etc) can be comprised of any effective type, e.g., ferromagnetic, supermagnetic, paramagnetic, and superparamagnetic. A preferred particle is comprised of iron oxide and polysaccharide. A preferred magnetic bead has a diameter which is less than the diameter of the cell which is to be captured, e.g., about 1-300 nm, about 5-200 mn, about 10-150 mn, preferably, about 20-150 nm, more preferably, about 50-120 nm. Preferably, the magnetic beads are of a sufficient size that they can form a coating around the cell, e.g., having more than one bead attached to the cell, such as about 10 beads, about 100 beads, about 1000, or about 100-1000 etc. These beads be manufactured or commercially obtained e.g., Milteni Biotech, Germany. See, also, U.S. Pat. Nos. 5,411,863; 5,543,289.

The first and second binding partner can then be added at the same time or sequentially. After each addition, optionally, an incubation period is utilized providing adequate time for the binding partner to attach to its substrate. Such times can be routinely determined. As a result of the above-mentioned steps, a cell-antigen-first binding partner-second binding partner combination is formed. The antigen-first binding partner-second binding partner combination can be referred to as a complex when at least these three components are joined together and attached to a cell. Preferably, the complex included a magnetic particle, e.g., when the second binding particle is attached to it. When a magnetic particle is included in the complex, separation can be achieved conventionally by a magnetic field. See, e.g., U.S. Pat. Nos. 5,541,072; 5,543,289; 5,238,810; 5,196,827; 4,731,337, e.g., by positive selection. For instance, in one embodiment, a chamber having an inlet and outlet is filled via the inlet with a sample. The sample contains, e.g., the cells (such as HIV-infected cells) coated with paramagnetic microspheres. A material which is capable of expressing a magnetic field surrounds the filled chamber. A magnetic field is applied to the column, retaining the cells coated with the paramagnetic beads, and allowing the uncoated cells to flow out through the outlet of the chamber. The infected, coated cells can be eluted by releasing the magnetic field. The chamber can comprise any material or matrix, including materials or matrices capable of expressing a magnetic field. Such technology is conventional. U.S. Pat. No. 5,411,863 describes an apparatus, system, and particles which can be used in the present invention.

As discussed, methods of the present invention relate to activating latently-infected cells by contacting such cells with an agent capable of activating an HIV virus integrated into the genome of the cells or incubating latently-infected cells under conditions effective to activate an HIV virus integrated into the genome of the cells. By the phrase, “activating an HIV virus,” it is meant that the agent and/or conditions induce the cell to express a characteristic of viral infection, such as infectious virus, reverse transcriptase, soluble antigen, cell-surface antigen such as gp120 or gp41, etc. In preferred embodiments of the invention, the agent and/or conditions are effective to elicit the expression of cell-surface gp120.

Any suitable viral activating agent can be used. A viral activating agent is any agent which can stimulate proviral latent DNA integrated into the genome to begin replication and production of infectious virus and/or cell-surface antigens, such as gp120 and/or gp41. For example, agents which are can induce active infection from a latent virus include, e.g., phorbol esters, such as phorbol myristate acetate (PMA) or; TNF-alpha; interleukins, such as IL-2, IL-12, IL-6, IL-15; cytokines; etc. Other agents can be identified routinely. For example, established cell lines harboring latent HIV-1, such as OM-10.1, U1, or Jurkat cells, can be treated with various amount of an agent to determine effective doses and conditions for eliciting productive infection.

To measure the latent viral load in resting cells, generally cells can be treated with an effective amount of an agent capable of activating a virus integrated into the genome of cells. The effective amount is any quantity of agent which is able to stimulate the cell under suitable conditions to cause a productive infection. Once productive infection results, cells expressing a surface-antigen, such as gp120 and/or gp41, can be isolated. Latent viral load can be expressed as cells per unit volume, such per ml. Alternatively, it can be expressed as an ED₅₀ when multiple samples are assayed for viral load.

Viral activation and methods of determining effective amounts can be performed in accordance with any suitable method. Various methods are described in, e.g., Kim et al., AIDS Res. Hum. Retroviruses, 20:1361-1366, 1996; Chun et al., Proc. Natl. Acad. Sci., 94:13193-13197, 1997; Tobiume et al., J. Gen. Viro., 79:1363-1371, 1998; Chun et al., J. Exp. Med., 188:83-91, 1998. Resting cells can be contacted directly with a viral activating agent, or pre-treated prior to contact. For instance, cells can be synchronized or arrested in the cell cycle, e.g., as desribed in Tobiume et al., 1998. Cells can also be precultured and then retested for cell-surface viral antigen, or other suitable characteristic of productive viral infection, to ensure that the population only comprises unifected and latently-infected cells. Agents can be used in any amount effective to activate latent virus, e.g., IL-2 (10-1000 U/ml, preferably about 100 U/ml); IL-1β (0.5-50 ng/ml, preferably about 5 ng/ml); IL-4 (0.3-30 ng/ml, preferably about 3 ng/ml); IL-6 (0.5-50 ng/ml, preferably about 5 ng/ml), and TNF-alpha (0.25-25 ng/ml, preferably about 2.5 ng/ml). After contact with the agent, cells can be incubated for various amounts time before assaying for the presence of antigen, such as gp120 and/or gp41, e.g, 8 hrs, 12 hrs, 16 hrs, 1-14 days, etc

In some circumstances, it may be desirable to culture purified resting cells in the absence of activating stimuli to allow for the degradation of labile, unintegrated forms of the virus. Cells can be cultured according to standard procedures for various time periods, e.g., 6 hrs or more, 12 hrs or more, 2 days, 4 days, 6 days, 8 days, etc., in order to allow the cells to become purged of any labile virus. After such culture period, it may be desirable to reselect the culture for the presence of gp120 or gp41 expressing cells.

The latent viral load can be correlated with the number of integrated proviruses, where the latter is measured conventionally as discussed above. The latent viral load of a patient can also be compared to the mentioned continuous cell lines which contain integrated provirus, e.g., OM-10.1, U1 or Jurkat cells. The latter can serve as a control, a standard, or a means of comparison.

### EXAMPLES

### Example 1

This assay is used to physically separate HIV-1 positive cells from a mixture of HIV-positive and HIV-negative cells. The HIV-positive cells are labeled with FITC-conjugated-HIV-1 monoclonal antibodies that are specific for gp120, a cell surface marker expressed when a cell is infected with HIV. Unbound antibody is removed and the cells are washed three times. The HIV-positive cells labeled with FITC-conjugated anti-HIV-1 antibody are further contacted with anti-FITC antibody conjugated to magnetic beads that are about 50 nanometers in diameter. The positive cells are now “tagged.” The magnetic beads do not interfere with standard fluorescent microscopy or flow cytometry quantification techniques. HIV-negative cells are not labeled in this process.

The labeled HIV-1 infected cells are loaded into a separation column and exposed to a magnetic field. The negative cells are eluted from the separation column while in the magnetic field and the positive cells are retained. The positive cells have now been “dragged.” The positive cells are eluted from the column by removing the column from the magnetic field. The cell separation kit achieves about 99% or greater purity of gp120/41-expressing cells from a mixture of HIV-1 positive and negative cells as scored by flow cytometry.

The aliquots of positive and negative HIV cells can now be analyzed (or simply counted) as desired. When using PBMCs, still viable HIV-1 positive cells may be further analyzed into lymphocyte subsets, cultured, or studied further prior to counting, using flow cytometric analysis of fluorescent miscroscopy.

### Example 2

A biopsy of tonsillar lymphoid tissue is removed from a patient treated with HAART. The tissue is macerated and filtered to produce a single cell suspension.

(1) Place 5-10×10⁶ cells into a 15 ml conical tube, count and centrifuge at 1000× g for 6 minutes at 4° C. Aspirate supernatant and re-suspend cell pellet in 5 ml of a suitable buffer solution.

(2) Centifuge, as in step (1), and aspirate supernatant. Protect tube and contents from light during steps (3) through (15).

(3) Add 5 μ1 of FITC-conjugated anti-HIV-1 monoclonal antibody specific for gp120, for every 3×10⁵ cells, to cell pellet and mix by gently pipetting. Incubate for 45 minutes at 4° C. on ice.

(4) Add 5 ml 1X PBS to cell suspension, resuspend, and centrifuge, as in step (1). Aspirate supernatant.

(5) Resuspend cell pellet in 5 ml. of 1X PBS. Centrifuge, as in step (1), and aspirate supernatant.

(6) Resuspend cell pellet in 2 ml of 1.0% paraformaldehyde. Incubate for 30 minutes at 4° C. on ice in dark. Centrifuge, as in step (1), and aspirate supernatant.

(7) Re-suspend cell pellet in 2.0 ml. buffer.

(8) Centrifuge, as in step (1) and aspirate supernatant from cell pellet.

(9) Take cell pellet from step (8) and re-suspend in 0.09 ml buffer. Maintain temperature of tube at 4° C. on ice.

(10) Add 10 μ1 anti-FITC magnetic bead conjugate to suspension in step (9).

(11) Dilute cell suspension with an additional 0.4 ml of diluted, degassed buffer prior to going to step (13).

(12) If quantitative results are desired, count cells again at this time.

(13) Equilibrate separation column.

(14) Pipette cell suspension from step (11) on to the separation column which is in magnetic separator from step (13) Let non-expressing cells pass through and collect in 12×75 mm culture tube. Wash 3 times with 1.0 ml of buffer to collect the non-expressing cells.

(15) Gently remove separation column from magnetic separator. Cap column tip and mix with vortex mixer. Remove cap and immediately place separation column on 12×75 mm culture tube. Pipette 3.0 ml of buffer on to separation column and flush out expressing cells leaving approximately 0.2 ml in column reservoir.

(16) Count cells from each aliquot collected and analyze by flow cytometry or other techniques, such as fluorescent microscopy.

### Example 3

Cells are depleted of cells expressing cell-surface gp120 as described in Example 2. The depleted cell population is sorted by FACS into HLA-DR⁺ and HLA-DR⁻ cells using anti-HLA-DR polyclonal antibodies conjugated to fluorescein. An aliquot of HLA-DR⁻ cells is taken and stained with rhodamine-conjugated anti-HLA-DR antibody and alkaline-phosphatase conjugated gp120 antibody and visually inspected to identify the presence and quantity of positive cells. The cell sorting procedure was repeated until about >99% of the cells were HLA-DR⁻ and negative for cell-surface viral antigen. This population is referred to as the resting cells.

The resting population is incubated a complete medium containing RPMI supplemented with 10% FCS, penicillin-streptomycin, and 1-glutamine in a tissue culture plate. Cytokines were added at the following concentrations: IL-2 (100 U/ml); IL-1μ(5 ng/ml); IL-4 (3 ng/ml); IL-6 (5 ng/ml) and TNF-alpha (2.5 ng/ml). Cultures were incubated in a 37° C. CO₂ incubator for at least 8 hrs. Aliquots of cells were tested for the presence of gp120 at 8 hrs, 16 hrs, 1 day, 2 day, 3 day, and 5 days using the method described in Examples 1 and 2 above.

IL-2 and TNF-alpha are potent activators of latent virus. De novo gp120 expression is observed in resting cells at least 1 day after exposure to the agents.

For other aspects of the polypeptides, antibodies, etc., reference is made to standard textbooks of molecular biology, protein science, and immunology. See, e.g., Abbas et al. (1997), Cellular and Molecular Immunology, W. B. Saunders and Co.; Davis et al. (1986), Basic Methods in Molecular Biology, Elsevir Sciences Publishing, Inc., New York; Molecular Cloning, Sambrook et al.; Current Protocols in Molecular Biology, Edited by F. M. Ausubel et al., John Wiley & Sons, Inc; Current Protocols in Human Genetics, Edited by Nicholas C. Dracopoli et al., John Wiley & Sons, Inc.; Current Protocols in Protein Science; Edited by John E. Coligan et al., John Wiley & Sons, Inc.; Current Protocols in Immunology; Edited by John E. Coligan et al., John Wiley & Sons, Inc.

From the foregoing description, one skilled in the art can easily ascertain the essential characteristics of this invention, and without departing from the spirit and scope thereof, can make various changes and modifications of the invention to adapt it to various usages and conditions.

Without further elaboration, it is believed that one skilled in the art can, using the preceding description, utilize the present invention to its fullest extent. The preceding preferred specific embodiments are, therefore, to be construed as merely illustrative, and not limiting of the remainder of the disclosure in any way whatsoever. The entire disclosure of all applications, patents and publications, cited above and in the figures are hereby incorporated by reference in their entirety, including, U.S. Ser. No. 09/139,633 filed Aug. 25, 1998; PCT/US97/18649, filed Oct. 15, 1997; U.S. Pat. Nos. 5,817,458; and 5,714,390.

1. A method of determining the latent viral load in a host infected with HIV comprising,
treating resting lymphoid mononuclear cells obtained from the host with an effective amount of an agent capable of activating an HIV virus integrated into the genome of the cells; and
detecting the expression of cell-surface gp120 after the cells have been treated with the agent, wherein the presence or amount of cells expressing cell-surface gp120 is a measure of latent viral load.

2. A method of claims 1, further comprising obtaining the resting lymphoid mononuclear cells by the steps of:
a) obtaining a sample cell population;
b) depleting the sample cell population of cells expressing cell-surface gp120; and
c) depleting sample cell population of cells expressing HLA-DR.

3. A method of claim 2, wherein the sample cells are depleted of gp120 expressing cells by the steps of:
a) contacting sample cells with gp120-specific antibodies, each conjugated to a capture moiety, under conditions effective for the antibodies to attach to gp120 on the surface of cells, thereby forming labeled-cells;
b) contacting the labeled-cells with capture moiety-specific antibody under conditions effective for the capture moiety-specific antibody to attach to the labeled-cells, thereby forming a complex-labeled cells; and
c) removing the complex-labeled cells, thereby depleting sample cells of gp120+ cells.

4. A method of claim 3, wherein the capture moiety-specific antibody is conjugated to magnetic particles.

5. A method of claim 3, wherein the capture moiety is FITC and the capture moiety-specific antibody is FITC-specific antibody conjugated to a magnetic bead.

6. A method of claims 4, wherein the magnetic particles are 10-100 nm in diameter.

7. A method of claims 5, wherein the magnetic particles are 10-100 nm in diameter.

8. A method of claims 3, wherein the removing is accomplished by a magnetic field acting on the magnetic particles.

9. A method of claim 2, further comprising:
separating CD4+ cells from the sample.

10. A method of claim 2, further comprising:
separating CD8+ cells from the sample.

11. A method of claim 2, wherein the depleting sample cell population of cells expressing HLA-DR is accomplished by flow cytometry cell sorting and said cells are labeled with a fluorochrome-labeled antibody specific-for HLA-DR.

12. A method of claim 1, wherein the tissue is lymphoid.

13. A method of claims 1, wherein the agent is phorbol ester or a cytokine.

14. A method of claim 1, wherein the measure of latent viral load is number of cells expressing gp120 after treating the resting with an effective amount of an agent capable of activating an HIV virus integrated into the genome of the cells.

15. A method of claim 1, wherein the measure of latent viral load is compared to an established cell line harboring latent HIV-1.

16. A method of claim 15, wherein the cell line is OM-10.1, U1, or Jurkat cells.

17. A method of treating a viral infection comprising
measuring the latent viral load in an HIV-infected patient; and
determining whether to administer to the patient an agent capable of activating an HIV virus integrated into the genome of a cell by the value of the latent viral load.
